(19) United States
(12) Patent Application Publication
(10) Pub. No.: US 2003/0096316 A1
(43) Pub. Date: May 22, 2003

(54) Use of apolipoprotein B as a single marker for evaluation of the risk or cardiovascular disease

(76) Inventor: Ingmar Wester, Turku (FI)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 09/989,681

(22) Filed: Nov. 21, 2001

(51) Int. Cl. G01N 33/53; G01N 33/567; G01N 33/542

(52) U.S. Cl. 435/7.2; 435/7.9

(57) ABSTRACT

Use of apolipoprotein B as a single marker for evaluation of the risk of cardiovascular disease.

### BACKGROUND OF THE INVENTION

Cardiovascular disease (CVD) is one of the major causes of death in western world. Elevated serum total and/or LDL cholesterol and triacylglycerol levels, as well as low ratio of HDL cholesterol to LDL cholesterol, are some of the major risk factors for CVD. Population studies have shown that CVD risk is directly related to increased concentrations in blood plasma of LDL cholesterol as well as triacylglycerols. Recently serum LDL cholesterol has been monitored as one of the major cholesterol-related risk factors for CVD. Serum LDL cholesterol levels can only be analyzed after tedious, cumbersome and expensive separation of serum lipoproteins by sequential flotation. Additionally serum LDL cholesterol can be calculated by the Friedewald equation (LDL cholesterol; mg/dl=total cholesterol−HDL cholesterol−triacylglycerols/5) only after the serum total and HDL cholesterol in addition to serum triacylglycerols have first been independently analyzed. This approach, although universally applied, has severe limitations. First it is based on three independent analysis increasing the overall costs and analytical variation. Secondly, blood sampling need to be taken after a 12 hour fasting and it can only be applied if serum triacylglycerol levels are below 400 mg/dl. Calculated serum LDL cholesterol concentrations normally differs substantially from LDL cholesterol concentrations measured after separation of LDL lipoprotein particles by sequential flotation with subsequent enzymatic measurement of the cholesterol content. Thus there is a definitive need for a fast, reliable and cost-effective approach for evaluating the efficacy of dietary regimen reducing the risk of CVD by reducing the amount of atherogenic lipoprotein particles in blood.

In most developed countries a substantial amount of the people have serum cholesterol levels not within the recommended levels. One of the first steps in improving the serum lipid profile is changes in life style including changes in diet and exercise. It seems, however, difficult to change dietary habits and to follow dietary recommendations, especially since following of the improvements by currently available methods are cumbersome and expensive. Thus it is important to make available means by which individuals can easily monitor improvements in the CVD risk in a simple and cost-effective way.

### SUMMARY OF THE INVENTION

The present invention relates to the use of serum apolipoprotein B (apo B) as a measurement of reduced risk of CVD when using dietary supplements and foods enriched with components having serum total and LDL cholesterol reducing effects. Especially it concerns the use of serum apo B as a measurement of reduced CVD risk due to oral intake of phytosterols and other nutraceuticals having a cholesterol-lowering effect. It further relates to the use of serum apo B measurement as a screening method for compounds and ingredients having beneficial health effects by reducing atherogenic lipoprotein particles in blood.

A method of determining a risk of cardiovascular disease according to the invention comprises obtaining a body fluid or cell sample from the subject; measuring a level of apo B in the sample; and correlating the level of apo B obtained in the measuring step with the risk of cardiovascular disease in the subject. The major improvement of the invention with respect to prior tests is that the apo B is the only sample component used in the test to correlate with the risk of cardiovascular disease. Preferably, other components of the sample usually used in testing for risk of cardiovascular disease (such as cholesterol and/or triacylglycerols) are not measured. If such components are measured for reasons unrelated to the present test, these measurements are not used to correlate with the risk of cardiovascular disease. Only apo B levels are used for this purpose.

A method of monitoring a risk of cardiovascular disease in a subject according to the invention comprises

obtaining an initial body fluid or cell sample from the subject;

determining a baseline level of apo B in the initial sample;

feeding to sthe subject a substance which reduces the apo B level in the subject;

obtaining an additional body fluid or cell sample from the subject;

measuring a level of apo B in the additional sample;

comparing the level of apo B obtained from the additional sample with the baseline level; and

correlating any change in apo B level obtained in the comparing step with the risk of cardiovascular disease in the subject.

As with the first described method, in this second method according to the invention the apo B is the only sample component used in the test for the comparing and correlating steps. If other components are measured, they are not used for the monitoring according to this second aspect of the invention.

A method of screening for candidate substances suspected of having a cardiovascular disease-reducing effect comprises

obtaining an initial body fluid or cell sample from a subject;

determining a baseline level of apo B in the initial sample;

feeding to said subject a substance suspected of reducing the apo B level in the subject;

obtaining an additional body fluid or cell sample from the subject;

measuring a level of apo B in the additional sample; and

comparing the level of apo B obtained from the additional sample with the baseline level, wherein a reduced apo B level in the additional sample is indicative that the substance has a cardiovascular disease-reducing effect.

As with the two above-described methods, this third method of the invention has as its key advantage the use of apo B as the only sample component used in the comparing step.

### DESCRIPTION OF THE PREFERRED EMBODIMENTS

Preferably, the body fluid sample is a blood, serum or plasma sample, and the cell sample is a buccal cell sample. The subject is preferably human.

In each of the described methods, preferably the determining and/or measuring steps comprise contacting the apo B with a signal-generating substance, generating a signal from the signal-generating substance, and measuring the signal. Preferably, the signal-generating substance is an antibody or antigen for the apo B, which is directly or indirectly labeled. Such antibodies or antigens are commercially available, or can be easily manufactured with routine skill in the art.

Body fluid or cell apo B levels can easily be measured by standardized, reliable analytical procedures (Rader D J. Hoeg J M. Brewer H B. Quantitation of plasma apolipoproteins in the primary and secondary prevention of coronary artery disease. Annals of Internal Medicine 1994, 120(12) 1012-1025; Schaefer E J. Lamon-Fava S. Cohn S D. Schaefer M M. Ordovas J M. Castelli W P. Wilson P W. Effects of age, gender, and menopausal status on plasma low density lipoprotein cholesterol and apolipoprotein B levels in the Framingham Offspring Study. Journal of Lipid Research 1994, 35(5) 779-792; and Jungner I. Marcovina S M. Walldius G. Holme I. Kolar W. Steiner E. Apolipoprotein B and A-I values in 147576 Swedish males and females, standardized according to the World Health Organization-International Federation of Clinical Chemistry First International Reference Materials. Clinical Chemistry 1998, 44(8 Pt 1) 1641-1649). Even non-fasting blood or other body fluid or cell samples can be used, thus providing a versatile approach for individuals to monitor the change in CVD risk due to the use of serum total and LDL cholesterol-lowering dietary regimen, especially those incorporating cholesterol-lowering nutraceuticals such as phytosterols and soy proteins.

Lipoproteins are produced in the liver and they transport cholesterol in blood and lymph. There are two main types, low-density lipoproteins (LDL's), often referred to as the “bad” cholesterol, and high-density lipoproteins (HDL's), referred to as “good” cholesterol. Apo B is synthesized in the liver as a component of very low-density lipoproteins (VLDL's) and remains with this particle as it circulates through the plasma. VLDL's are the precursors to LDL's. The cholesterol content of LDL lipoprotein particles can vary, but there is only one apo B per LDL particle. Therefore, the measurement of apo B provides a measure of the number of lipoprotein particles containing that protein. Thus, the level of apo B is a marker for the number of atherogenic particles (mainly LDL and VLDL lipoprotein particles) in the blood and lymph.

LDL lipoprotein particles are largely responsible for the atherosclerotic buildup of fatty deposits on the blood vessels walls, a major factor in CVD and, thus, apo B is an independent predictor of CVD risk. It can therefore be used as a single marker for evaluating the risk of CVD in a reliable way.

Food products enriched with components having cholesterol-lowering effect beyond normal nutrition (i.e., “nutriceuticals”) have been commercially available for some time. Representative examples are food products enriched with soybean protein or phytosterols. Soy protein containing foods and dietary supplements have been boosted by the approval of a health claim by FDA. The current literature and especially FDA's interim approval of health claim for phytosterols have increased the interest of the food industry in supplementing foods with phytosterols. For example, stanol fatty acid esters and the cholesterol lowering effects thereof are disclosed in U.S. Pat. No. 5,502,045, as well as a suitable method for their preparation. Dietary intake of 2 to 3 g/day of plant stanols is reported to lower serum LDL cholesterol levels in man up to 14%, thus reducing the risk of coronary heart disease. Indeed, many such food items have recently been introduced into the market. It is assumed that new phytosterol containing foods will appear into the market rapidly.

As used here, the term “phytosterol” refers to any sterol defined as following and having lipid profile improving effect: both sterols and saturated sterols i.e. stanols in either their free form or esterified e.g. with fatty acids (2-24 carbon atoms, saturated, monounsaturated or polyunsaturated, including also special fatty acids such as conjugated fatty acids, e.g. CLA, and EPA and DHA), hydroxybenzoic acids, hydroxycinnamic acids (ferrulic or coumaric acids), other organic acids such as e.g. di- or tricarboxylic acids and/or hydroxy acids or with any combination of the said acids. Any combinations of the free and various esterified forms are also included.

The sterols include here 4-desmethyl sterols, 4-monomethyl sterols and 4,4-dimethyl sterols (triterpene alcohols) and the stanols include 4-desmethyl stanols, 4-monomethyl stanols and 4,4-dimethyl stanols. Typical 4-desmethyl sterols are sitosterol, campesterol, stigmasterol, brassicasterol, 22-dehydrobrassicasterol, Δ5-avenasterol. Typical 4,4-dimethyl sterols are cycloartenol, 24-methylenecycloartenol and cyclobranol. Typical stanols are sitostanol, campestanol and their 24-epimers, cycloartanol and saturated forms obtained by saturation of e.g. triterpene alcohols (cycloartenol, 24-methylenecycloartenol and cyclobranol). The phytosterols include all possible blends of any of these sterols and/or stanols as well as any mentioned individual sterol or stanol.

As used here the term “nutraceutical” refers to compounds or ingredients having a beneficial health effect. The term “cholesterol-lowering nutraceutical” refers to a compound/ingredient or compounds/ingredients reducing serum total and LDL cholesterol when ingested as such, as part of dietary supplements or foods. Typical “cholesterol-lowering nutraceuticals” are, but not limited to, phytosterols, soyproteins and other proteins having a cholesterol lowering effect, modified or un-modified, dietary fibres such as β-glucan and psyllium or any combinations of such cholesterol-lowering nutraceutical.

When performing the present invention the stanol fatty acid esters are the most preferable cholesterol-lowering nutraceutical based e.g. on their physiological effects, chemical stability and easy workability.

I claim:

1. A method of determining a risk of cardiovascular disease in a subject, comprising
obtaining a body fluid or cell sample from the subject;
measuring a level of apolipoprotein B (apo B) in said sample; and
correlating said level of apo B obtained in said measuring step with the risk of cardiovascular disease in the subject, wherein the apo B is the only sample component used in said correlating step.

2. The method of claim 1, wherein the body fluid sample is a blood, serum or plasma sample.

3. The method of claim 1, wherein the cell sample is a buccal cell sample.

4. The method of claim 1, wherein said measuring step comprises contacting the apo B with a signal-generating substance, generating a signal from the signal-generating substance, and measuring the signal.

5. The method of claim 4, wherein the signal-generating substance is an antibody or antigen for the apo B, which is directly or indirectly labeled.

6. The method of claim 1, wherein the apo B is the only sample component measured in said measuring step.

7. The method of claim 1, wherein, in said measuring step, any measurement of cholesterol is avoided.

8. The method of claim 1, wherein, in said measuring step, any measurement of triacylglycerols is avoided.

9. A method of monitoring a risk of cardiovascular disease in a subject, comprising
obtaining an initial body fluid or cell sample from the subject;
determining a baseline level of apolipoprotein B (apo B) in said initial sample;
feeding to said subject a substance which reduces the apo B level in the subject;
obtaining an additional body fluid or cell sample from the subject;
measuring a level of apolipoprotein B (apo B) in said additional sample;
comparing said level of apo B obtained from said additional sample with said baseline level; and
correlating any change in apo B level obtained in said comparing step with the risk of cardiovascular disease in the subject, wherein the apo B is the only sample component used in said comparing and correlating steps.

10. The method of claim 9, wherein the body fluid sample is a blood, serum or plasma sample.

11. The method of claim 9, wherein the cell sample is a buccal cell sample.

12. The method of claim 9, wherein said determining and/or measuring steps comprise contacting the apo B with a signal-generating substance, generating a signal from the signal-generating substance, and measuring the signal.

13. The method of claim 12, wherein the signal-generating substance is an antibody or antigen for the apo B, which is directly or indirectly labeled.

14. The method of claim 9, wherein the apo B is the only sample component measured in said determining and/or measuring steps.

15. The method of claim 9, wherein, in said determining and/or measuring steps, any measurement of cholesterol is avoided.

16. The method of claim 9, wherein, in said determining and/or measuring steps, any measurement of triacylglycerols is avoided.

17. The method of claim 9, wherein the substance is a cholesterol-lowering nutraceutical.

18. The method of claim 17, wherein the cholesterol-lowering nutraceutical is one or more substances selected from the group consisting of a phytosterol, soy protein, β-glucan and psyllium.

19. The method of claim 9, wherein the substance is a phytosterol.

20. The method of claim 19, wherein the phytosterol comprises a stanol fatty acid ester.

21. A method of screening for candidate substances suspected of having a cardiovascular disease-reducing effect, comprising
obtaining an initial body fluid or cell sample from a subject;
determining a baseline level of apolipoprotein B (apo B) in said initial sample;
feeding to said subject a substance suspected of reducing the apo B level in the subject;
obtaining an additional body fluid or cell sample from the subject;
measuring a level of apolipoprotein B (apo B) in said additional sample; and
comparing said level of apo B obtained from said additional sample with said baseline level, wherein a reduced apo B level in said additional sample is indicative that the substance has a cardiovascular disease-reducing effect, and wherein the apo B is the only sample component used in said comparing step.

22. The method of claim 21, wherein the body fluid sample is a blood, serum or plasma sample.

23. The method of claim 21, wherein the cell sample is a buccal cell sample.

24. The method of claim 21, wherein said determining and/or measuring steps comprise contacting the apo B with a signal-generating substance, generating a signal from the signal-generating substance, and measuring the signal.

25. The method of claim 24, wherein the signal-generating substance is an antibody or antigen for the apo B, which is directly or indirectly labeled.

26. The method of claim 21, wherein the apo B is the only sample component measured in said determining and/or measuring steps.

27. The method of claim 21, wherein, in said determining and/or measuring steps, any measurement of cholesterol is avoided.

28. The method of claim 21, wherein, in said determining and/or measuring steps, any measurement of triacylglycerols is avoided.
